SEQUENCE LISTING (14 sequences)

SEQ ID NO: 1 - DNA, 37 bp, Artificial Sequence
  PCR primers
gtctcaggtaccttccaccatggcggcggcggttcgg

SEQ ID NO: 2 - DNA, 30 bp, Artificial Sequence
  PCR primers
gatcatcgatgttattgtatggtaacatgg

SEQ ID NO: 3 - DNA, 20 bp, Artificial Sequence
  PCR primers
cagcatcacctctggtaccc

SEQ ID NO: 4 - DNA, 27 bp, Artificial Sequence
  PCR primers
cccgaattccggggcgaacgccggacg

SEQ ID NO: 5 - DNA, 21 bp, Artificial Sequence
  PCR primers
ctgccactcgatatgaacccg

SEQ ID NO: 6 - DNA, 23 bp, Artificial Sequence
  PCR primers
tagatccttaatggtctcaaccg

SEQ ID NO: 7 - DNA, 120 bp, Artificial Sequence
  PCR primers
ccggtaccgggttgtggcagccagtcacgtgcccgccgcgtagccacacctctgctcctcagagcaatgtcaagcggtcacgtgtgatagcaacagatcacgtggctgccatcgcccctc

SEQ ID NO: 8 - DNA, 117 bp, Artificial Sequence
  PCR primers
atgaattccggacgttctgggcacgtgaccgccacccatgcgctgaggggcggacaggaggtgcttcgactgggaggagggcgaagagtgtaagggggcggaggggcgatggcagcc

SEQ ID NO: 9 - DNA, 120 bp, Artificial Sequence
  PCR primers
ccggtaccgggttgtggcagccagtcacctgcccgccgcgtagccacacctctgctcctcagagcaatgtcaagcggtcacctgtgatagcaacagatcacctggctgccatcgcccctc

SEQ ID NO: 10 - DNA, 120 bp, Artificial Sequence
  PCR primers
atgaattccggacgttctgggcaggtgaccgccacccatgcgctgaggggcggacaggaggtgcttcgactgggaggagggcgaagagtgtaagggggcggaggggcgatggcagccagg

SEQ ID NO: 11 - DNA, 46 bp, Artificial Sequence
  PCR primers
gcggatccgcggccgccttccaccatggctacctctcgatctgagc

SEQ ID NO: 12 - DNA, 29 bp, Artificial Sequence
  PCR primers
cggtcgactcactccggattaccttcatc

SEQ ID NO: 13 - DNA, 443 bp, Homo sapiens
gtggagcgaaaaggtgacagcatcacctctggtaccccaactcccaccccctccccaatgcagacaggctgaaagaccggtagtgagactggagttcagccttcagaccggtagtgagacaatccttcagccgggagttgggctctgggtggcctaggttgccatggcaccgcctcgggctccaccctctcttgtccccctcaccagctccccccctgcagcgggggttgtggcagccagtcacgtgcccgccgcgtagccacacctctgctcctcagagcaatgtcaagcggtcacgtgtgatagcaacagatcacgtggctgccatcgcccctccgcccccttacactcttcgccctcctcccagtcgaagcacctcctgtccgcccctcagcgcatgggtggcggtcacgtgcccagaacgtccggcgttcgccccgccc

SEQ ID NO: 14 - DNA, 415 bp, Mus musculus
gtggagtggaaatgttacagcaccatttctggcaccaggatctcccactaacacctcgacctcccgcccactcagagacccatagtgagactgaagatgcgctctggctcggccttgagcccagagttgagagctggttggcccggttgccatgacaccgccttgtgctccaccctctcgcccccacacaccccctcggcagtcagggtatggcagccagtcacgtgccacacagcgtaaccacacctctgcttcccagcgcaaagtcaaggggtcacgtgggatagcaacaggtcacgtggccgtcagccccgcccccttccccaccacacccctcccatcaaagcagcccgggttgcccactgcgcaagggtgaagatcacgtgtccagaacgtccggcgcccgcccccgccc

(19) United States
(12) Patent Application Publication
(10) Pub. No.: US 2002/0111289 A1
(43) Pub. Date: Aug. 15, 2002

(54) CDK4 is a target of c-MYC

(76) Inventors: Bert Vogelstein, Baltimore, MD (US); Kenneth W. Kinzler, Bel Air, MD (US); Heiko Hermeking, Baltimore, MD (US)

Correspondence Address: BANNER & WITCOFF, 1001 G STREET N W, SUITE 1100, WASHINGTON, DC, 20001, US

(21) Appl. No.: 09/780,566

(22) Filed: Feb. 12, 2001

Related U.S. Application Data
(60) Provisional application No. 60/181,930, filed on Feb. 11, 2000.

(51) Int. Cl. A61K 31/00; C12Q 1/68; C12Q 1/00; C12N 15/74; C12N 5/06

(52) U.S. Cl. 514/1; 435/6; 435/4; 435/325; 435/320.1

(57) ABSTRACT

The prototypic oncogene c-MYC encodes a transcription factor, which can drive proliferation by promoting cell cycle re-entry. However, the mechanisms through which c-MYC achieves these effects have been unclear. Using serial analysis of gene expression (SAGE), we have identified the cyclin dependent kinase 4 (CDK4) gene as a transcriptional target of c-MYC. c-MYC induced a rapid increase in CDK4 mRNA levels through four highly conserved c-Myc binding sites (MBS) within the CDK4 promoter. Cell cycle progression is delayed in c-MYC-deficient RAT1 cells, and this delay was associated with a defect in CDK4 induction. Ectopic expression of CDK4 in these cells partially alleviated the growth defect. Thus, CDK4 provides a direct link between the oncogenic effects of c-MYC and cell cycle regulation.

### TECHNICAL FIELD OF THE INVENTION

This invention is related to cancer therapeutics and means of identifying new agents for treating cancers.

### BACKGROUND OF THE INVENTION

The proto-oncogene c-MYC has been implicated in a variety of human and experimental tumors (for review see: 1-4). In some cases, the overexpression of c-MYC can be traced to genetic alterations of the oncogene itself, while in others this dysregulation is due to genetic defects in upstream regulators of c-MYC expression. In either case, the ability of c-MYC to promote proliferation through cell cycle re-entry appears critical to its oncogenic function. Accordingly, expression of c-MYC is induced by a variety of mitogens and repressed under conditions of growth arrest. Furthermore, ectopic c-MYC expression can in some cases promote re-entry of resting cells into the cell cycle and facilitate proliferation in the absence of external growth factors (5).

The c-MYC gene encodes a transcription factor of the helix-loop-helix leucine zipper class (for review see 1, 2). C-MYC binds to E-boxes (CACGTG) in the vicinity of target genes which are then activated. The DNA binding activity requires dimerization with another helix-loop-helix leucine zipper protein called Max. Max can also interact with transcriptional repressors such as Mad and Mxil which presumably down-regulate expression of c-MYC target genes. Despite many advances and identification of a number of potential c-MYC target genes, the direct mediators of c-MYC's effects on cell cycle re-entry have not yet been identified. There is a continuing need in the art to identify the components of the cellular machinery which are dysregulated in cancers and which are susceptible to therapeutic interventions.

### SUMMARY OF THE INVENTION

It is an object of the present invention to provide a reporter construct useful for drug screening and identification.

It is another object of the present invention to provide a host cell useful for drug screening and identification.

It is an object of the present invention to provide a method to screen test compounds for anti-cancer activity.

It is an object of the present invention to provide an isolated and purified nucleic acid molecule.

It is an object of the present invention to provide a method of inhibiting the growth of tumor cells.

These and other objects of the invention are provided by one or more of the embodiments described below. In one embodiment, a reporter construct is provided. The reporter comprises an upstream region of a mammalian CDK4 gene transcription start site comprising at least four c-MYC binding sites and a coding sequence for a reporter protein. The upstream region is upstream of the coding sequence. The upstream region and coding sequence are operably linked so that a wild-type c-MYC upon binding to the upstream region activates transcription of the coding sequence.

According to another embodiment of the invention a host cell is provided. The host cell comprises a reporter construct as described above and a c-MYC protein. The c-MYC protein binds to the reporter construct and activates transcription of the coding sequence for the reporter protein.

According to still another aspect of the invention a method is provided for screening test compounds for anti-cancer activity. A c-MYC protein is contacted in the presence of a test compound with a reporter construct as described above. Expression of the reporter protein is monitored. A test compound which decreases expression of the reporter protein is a candidate anti-cancer agent.

Also provided by the present invention is an isolated and purified nucleic acid molecule. The molecule comprises at least one copy of a region upstream of a human CDK4 gene transcriptional start site. The region comprises at least four c-MYC binding sites comprising the sequence CACGTG. The nucleic acid molecule does not contain the CDK4 coding sequence.

According to another aspect of the invention another method is provided for screening test compounds for anti-cancer activity. A c-MYC protein is contacted in the presence of a test compound with a nucleic acid molecule as described above. Binding of c-MYC protein to the nucleic acid molecule is monitored. A test compound which decreases binding of c-MYC to the nucleic acid molecule is identified as a candidate anti-cancer agent.

Another embodiment of the invention provides a method of inhibiting the growth of tumor cells. Tumor cells which comprise a genetic alteration which causes c-MYC overexpression are contacted with an agent which inhibits CDK4 enzymatic activity. Tumor cell growth is thereby inhibited.

According to yet another aspect of the invention a method of screening compounds to identify those which have anti-cancer activity is provided. A cell which has a genetic alteration which dysregulates c-MYC expression is contacted with a test compound. Activity of CDK4 in the cell is measured. A test compound which inhibits activity of CDK4 is identified as a candidate agent with anti-cancer activity.

These and other embodiments provide the art with new targets for therapeutic intervention and drug discovery for cancers.

### BRIEF DESCRIPTION OF THE DRAWINGS

FIGS. 1A-1D show the effects of ectopic c-MYC and MadMyc expression on cell cycle distribution and CDK4 mRNA/protein levels.

FIG. 1A. Flow cytometric analysis of serum starved HUVEC cells (48 hours in 0.5% serum) which were infected with the indicated viruses and maintained in 0.5% serum (−serum) or restimulated by addition of 2% serum (+serum). Cells were harvested 12 (lower panel) or 24 hours (upper panel) after viral infection and subjected to flow cytometric analysis as described in (8).

FIG. 1B. Northern blot analysis with RNA (2.5 mg) from HUVEC cells serum starved (0.5%) for 24 hours and then subjected to the serum stimulation (2%) and/or adenoviral infection as indicated. Membranes were hybridized with a probe for CDK4 or a control probe for Laminin mRNA.

FIG. 1C. Western blot analysis of lysates from serum starved HUVEC cells (48 h in 0.5% serum) infected with Ad-Myc, Ad-GFP or serum stimulated and harvested at the indicated times. Membranes were probed with a CDK4 specific antibody (see Materials and Methods).

FIG. 1D. Northern blot analysis with RNA from a human B-cell line (P493-6) after activation of a conditional c-MYC allele. P493-6 cells harbor a c-MYC gene under control of a tetracycline-responsive element (24).

FIGS. 2A-2E show MYC binding sites (MBS) in the CDK4 promoter.

FIG. 2 A. Map of the human CDK4 gene indicating the position of E-boxes (MBS) in the promoter of the human CDK4 gene (black rectangles: MBS1-5). Grey shading represents the CDK4 open reading frame (ORF). The arrow indicates the transcription start site (TSS).

FIG. 2B. Alignment of the human and mouse CDK4 promoter sequence upstream of the TSS. Identical residues are shaded black and the identical MBS are shaded gray.

FIG. 2C. Gel electrophoretic mobility shift assay. Oligonucleotides encompassing the first 200 bp upstream of the TSS depicted in (B) containing either wildtype (wt) or mutant (mt) MBS were end-labeled with [γ-³²P] ATP and incubated with combinations of in vitro translated MYC and MAX proteins (38). DNA-protein complexes were separated by electrophoresis and detected as “shifts” from the position of the free probe. Addition of an antibody (Ab) directed against an HA-epitope engineered to the C-terminus of MAX was able to generate a “supershifted” band as indicated by the asterisk. Unlabeled oligonucleotides (40×excess) were used as competitors in some reactions. Luciferase activity of CDK4 promotor constructs was measured in Rat1 cells cotransfected with the indicated reporter and a β-galactosidase expressing vector as control.

FIG. 2D. Luciferase activity is presented as the average of three separate experiments with standard deviation as error bars.

FIG. 2E. Luciferase activity of indicated CDK4 promotor constructs (MBS1-4 or mutMBS1-4) was measured in NIH3T3 cells cotransfected with empty vector (Control) or the indicated amounts (mg) of expression vectors for wild type (WT) c-Myc or mutant c-Myc (16). Luciferase activity was measured 48 hours after transfection and presented as relative activity normalized to the control activity of the wild type promotor (MBS1-4). Values are the average of four determinations with the standard deviation as error bars.

FIGS. 3A-3B. Requirement of c-Myc for normal induction of Cdk4 after serum-stimulation.

FIG. 3A. RAT1 c-Myc+/+(TGR-1) and Rat1 c-Myc−/−(HO15.19) were serum-starved for 48 hours in DMEM containing 0.25% calf serum. RAT1 and RAT1 c-Myc−/−were restimulated with 10% calf serum/DMEM and RNA lysates prepared at the indicated times. Northern blot analysis was performed with a probe for Rat Cdk4 and Gap-DH as an internal control. Relative Cdk4 mRNA levels were determined by quantitating the hybridization signal using a Phosphorlmager followed by correction for the number of cells loaded using the internal Gap-DH standards.

FIG. 3B. RAT1 c-Myc+/+(TGR-1) and Rat1 c-Myc−/−(HO15.19) were serum-stimulated as described in FIG. 3A and protein lysates prepared at the indicated time. Western blot analyses were performed with antibodies against CDK4, Cyclin D1, and α-Tubulin.

FIGS. 4A-4B. Growth enhancement of c-Myc-deficient cells by ectopic CDK4 expression.

FIG. 4A. Western blot analysis of CDK4 expression in c-Myc-deficient RAT1 cell infected with a CDK4 encoding retrovirus and a gene conferring hygromycin resistance. CDK4-P1, -P2, and -P3 represent pools of hygromycin resistant c-Myc−/−cells. “CDK4” refers to the endogenous CDK4.

FIG. 4B. The pools from FIG. 4A were analyzed for growth rates. Cells were seeded in DMEM containing 10% calf serum and counted at 24-hour intervals. Each time point represents the average of two independent experiments.

FIG. 5. Correlation between c-MYC and CDK4 mRNA in colorectal tumors. Northern blot analysis with RNA isolated from normal colonic epithelial cells and tumor cells derived from 3 different patients.

### DETAILED DESCRIPTION OF THE INVENTION

It is a discovery of the applicants that CDK4 gene expression is directly regulated by c-MYC. c-MYC is known to drive cellular proliferation by promoting cell cycle re-entry. It is genetically dysregulated in a variety of specific cancers. CDK4 provides a direct link between the oncogenic effects of c-MYC and cell cycle regulation.

Based on this direct link, one can screen test compounds for anti-cancer activity in ways not previously envisioned. For example, one can screen compounds for those which alter transcriptional responsiveness of an upstream region of a mammalian CDK4 gene to c-MYC. Since the region appears to be highly conserved among mammalian species, the region can be derived from any mammalian species, including human, mouse, rat, cow, hamster, guinea pig, monkey, ape, chimpanzee, etc. Transcriptional responsiveness can be monitored by using a reporter gene which upon transcription/translation yields a reporter protein. A test compound which decreases expression of the reporter protein is a candidate anti-cancer agent. Transcriptional responsiveness can be measured in an in vitro transcription/translation system, or in a cell which harbors a reporter construct which comprises the upstream region and a coding sequence for the reporter protein. The reporter protein can be any whose expression is easy to monitor. It can be an enzyme, or a fluorescent protein, for example. Many suitable reporter proteins are known in the art. Appropriate means of monitoring reporter proteins are known in the art. For example, assays are known which can be used to conveniently monitor enzymes. Fluorescence detection techniques are known in the art for detecting fluorescent proteins.

A reporter construct, as mentioned above, has an upstream region of a mammalian CDK4 gene and a coding sequence of a reporter protein. The reporter gene is preferably not the CDK4 protein. The two elements of the reporter construct are operably linked so that a wild-type c-MYC activates transcription of the coding sequence upon binding to the upstream region. The upstream region is typically at least 200 bp and contains at least four c-MYC binding sites. These contain CACGTG motifs.

Host cells which contain the reporter construct are useful for cell-based drug screening assays. Any host cell can be used which is compatible with the reporter construct. Typically certain vectors can be replicated in certain host cells. Preferably the host cell will express a c-MYC protein which will bind to the upstream region contained within the reporter construct. More preferably the host cell expresses more c-MYC protein than a normal cell. Even more preferably the host cell is a tumor cell which is genetically altered so that it expresses more c-MYC protein than a normal cell.

The amount of decrease of expression will vary from compound to compound tested. Larger decreases are believed to be indicative of greater ultimate therapeutic usefulness. However, other factors are also important in evaluating ultimate therapeutic usefulness, as are well known in the art. These include solubility, cellular uptake, serum stability, side effects, toxicity.

Isolated and purified nucleic acid molecules which contain the upstream region of human CDK4 typically do not contain the amino acid-coding sequence of CDK4. Such molecules can be attached to a solid support and used inter alia for purifying c-MYC, and for assaying the strength and/or amount of binding of c-MYC.

Test substances can be contacted with a c-MYC protein and their effect on the protein's binding to a nucleic acid molecule containing the CDK4 upstream region can be monitored. Binding can be assessed according to any method known in the art, including but not limited to a gel electrophoresis mobility shift assay (as described below), using antibodies, and on a column of immobilized nucleic acids.

Tumor cell growth can be inhibited either in vitro or in vivo by administration of an agent which inhibits CDK4 enzymatic activity. Preferably the tumor cell will have a genetic alteration which causes c-MYC overexpression. Such alterations are known to occur in Burkitt's Lymphoma, neuroblastoma, and colon cancer. Known genetic alterations which affect such dysregulation include a t8;14 translocation, amplification of c-MYC, and mutations in APC or β-catenin. Agents which can be used to inhibit the enzymatic activity of CDK4 include any which are known in the art. Protein p16 and truncated versions of it as well as p18 inhibit CDK4 and can be used to inhibit tumor cell growth of cells which have genetic alterations which cause c-MYC overexpression. Fahraeus et al., Oncogene 1998 5:587-96 (disclosing p16 derivatives). See also Kubo et al, Clin. Cancer Res. 1999, 5:4279-86 (disclosing 3-amino thioacridone and its structural homologs). Agents can be administered by any mode known in the art which retains agent activity and provides access to the cancer cells. These include without limitation oral, intravenous, intraperitoneal, subcutaneous, intramuscular, intrathecal.

Cells which have a genetic alteration which dysregulates c-MYC expression can also be used to screen for potential anti-cancer drugs. Test compounds can be contacted with such cells and their effects on the cells' CDK4 enzymatic activity can be monitored. A test compound which inhibits CDK4 activity is identified as a candidate agent with anti-cancer activity. Methods for assaying for CDK4 enzymatic (kinase) activity are known in the art and any such method can be used. See for example Li J, et al., Biochemistry, 2000, 39:649-657.

The above disclosure generally describes the present invention. A more complete understanding can be obtained by reference to the following specific examples which are provided herein for purposes of illustration only, and are not intended to limit the scope of the invention.

### EXAMPLE 1

This example demonstrates that c-MYC expression is necessary but not sufficient for cell cycle re-entry.

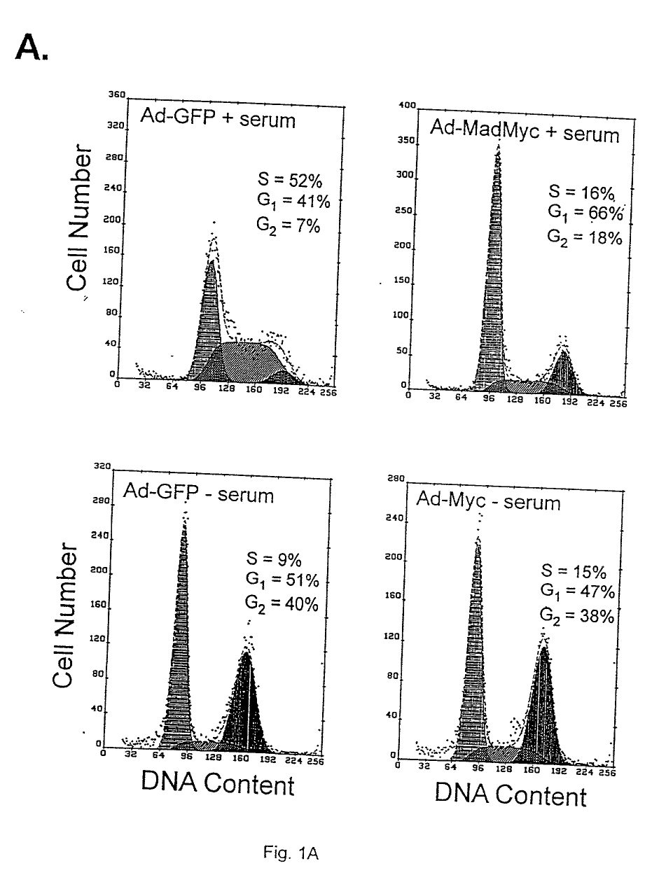

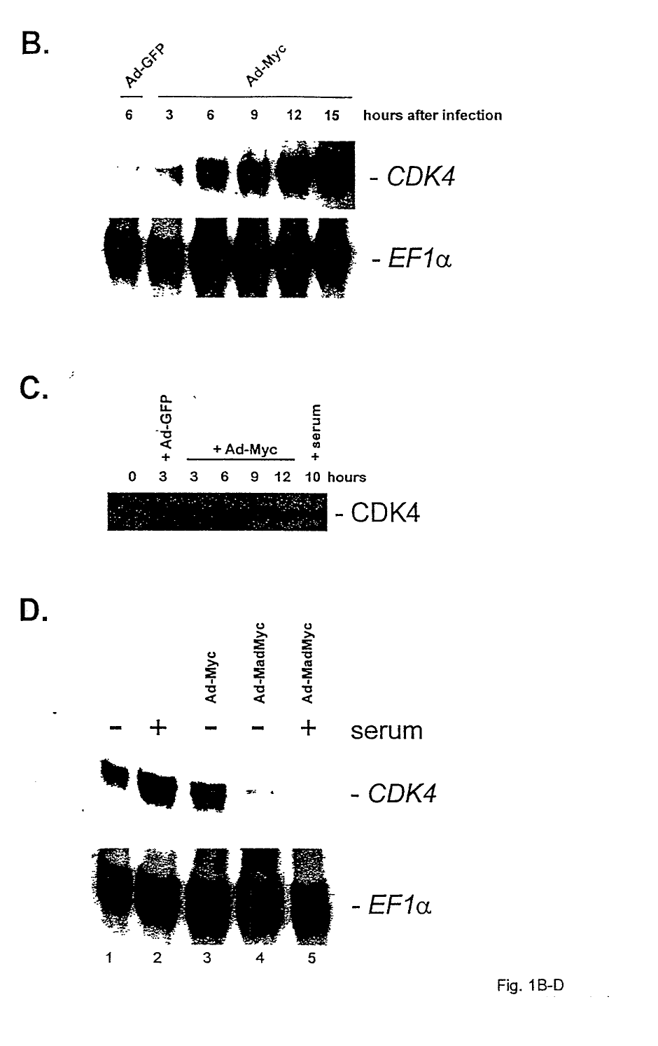

Infection of human umbilical vein cord (HUVEC) cells with an adenovirus containing a dominant negative mutant of c-MYC (MadMyc,(10)) prevented their serum-induced re-entry into the cell cycle (FIG. 1A). Infection with an adenovirus containing a wild type c-MYC gene did not efficiently induce re-entry in the absence of serum (FIG. 1A). In combination, these results suggest that c-MYC expression is necessary but not sufficient for HUVEC cell cycle re-entry. Furthermore, this system provided a way to potentially identity the genes regulated by c-MYC in the absence of incidental changes associated with proliferation.

Cell Culture, Medium and Reagents. Human umbilical vein cord cells (HUVEC) and their respective media were obtained from Clonetics (San Diego, Calif.).

Adenovirus Generation. High titer adenovirus expressing c-MYC or MadMyc was generated using the AdEasy system as described (8). In brief, a fragment containing the CMV-promoter and a human c-MYC cDNA fused to an HA-epitope-tag was excised from the construct HH67 (9) using the restriction enzymes Xho I and Hind III and inserted into the shuttle vector pAdTrack. To generate an HA-epitope tagged MadMyc cDNA, the previously described MadMyc encoding plasmid (10) was employed as a template in a PCR using the primers 5′-GTCTCAGGTACCTTCCACCATGGCGGCGGCGGTTCGG-3′ and 5′-GATCATCGATGTTATTGTATGGTAACATGG-3′. The resulting fragment was cut with Kpn I and Cla I and ligated into the HH67 vector (see above) digested with the same enzymes. A fragment containing the CMV-promoter and the MadMyc-ORF was then transferred to pAdTrack. After recombination with the vector pAdEasy, high titer virus was generated in 911 and 293 cells. Viruses were purified via a CsCl gradient and the effective titer was determined by the frequency of GFP positive cells after infection. The efficiency of the infection was normalized to the frequency and intensity of GFP positive cells.

### EXAMPLE 2

This example demonstrates the association of expression of CDK4 with c-MYC.

Serial analysis of gene expression (SAGE) was used to determine which genes are induced by expression of c-MYC in these human cells. SAGE was performed on serum-starved HUVEC cells 12 hours after infection with either a c-MYC-expressing virus (Ad-Myc) or a control virus containing the gene for green fluorescent protein (Ad-GFP). The most intriguing c-MYC induced transcript in terms of cell cycle regulations was that encoding the cyclin dependent kinase 4 (CDK4) (17). This transcript was of particular interest as ectopic expression of CDK4 had been previously shown to mimic some of the effects of c-MYC overexpression. For example, expression of CDK4 or c-MYC is sufficient to prevent the cell cycle arrest associated with serum-starvation (5,14), exposure to TGF-β(18,19), or ectopic expression of p53 (20,21). Likewise, c-MYC and CDK4 genes can both immortalize primary cells (22,23).

Induction of CDK4 mRNA was detectable as early as 6 hours after infection with Ad-Myc and increased 3-4 fold by 15 hours (FIG. 1B and data not shown). This increase in CDK4 mRNA was accompanied by an induction of CDK4 protein (FIG. 1C). CDK4 mRNA was also induced after addition of serum to serum-starved cells (FIG. 1B, compare lanes 1 and 2). This induction of CDK4 by serum was dependent on c-MYC, as adenoviral expression of dominant-negative mutant MadMyc prevented the induction of CDK4 mRNA after serum-stimulation (FIG. 1B, compare lanes 2 and 5). Expression of MadMyc also led to a reduction in the low level of CDK4 mRNA present in serum-starved cells (FIG 1B, compare lanes 1 and 4).

In order to test whether other cell types displayed c-MYC regulation of CDK4, human primary B-cells engineered with a tetracycline inducible c-MYC gene were employed (24). Induction of c-MYC RNA was detectable 4 hours after removal of tetracycline. Induction CDK4 mRNA lagged 1 hour behind the c-Myc induction (FIG. 1D). Induction of CDK4 protein lagged 2 hours behind the induction of CDK4 mRNA (Schuhmacher et al., unpublished data).

Taken together, these results suggested that c-MYC directly regulates CDK4 mRNA expression.

Sage Analysis. Total RNA was harvested 12 hours after Ad-Myc or Ad-GFP infection of HUVEC cells which had been arrested by serum starvation for 48 hours. SAGE was performed as described (11,12) and a total of 92,478 tags representing approximately 8,500 different transcripts were analyzed to identify candidate c-Myc induced genes.

Northern Blot Analysis. Total RNA was prepared by CsCl gradient ultracentrifugation of guanidine isothiocyanate-lysed cells as described (11). Probes directed against the 3′ untranslated region of the respective mRNAs were generated by PCR using ESTs as templates and subsequent gel-purification. Hybridizations were performed in QuickHyb following the manufacturer's instructions (Stratagene).

Western Blot Analysis. For Western blot analysis, cells were lysed in 2×Laemmli buffer. Proteins were separated on SDS/polyacrylamide gels (Novex) and transferred to nitrocellulose membranes (Millipore). Membranes were preblocked in 5% milk/TBS for 30 minutes and then probed with different primary antibodies diluted in 5% milk/TBS/0.05% Tween 20 for 60 minutes and then for 30 minutes with an HRP-coupled secondary antibody. After washing the membranes for 30 minutes in TBS/0.05% Tween 20, ECL (enhanced chemiluminescence) detection was performed according to the manufacturer's instructions NEN). Primary antibodies used for detection were AB-1/DCS-35 (Neomarkers) for cdk4, A-12 (Santa Cruz) for cyclin D1, rat a-HA (Cat# 1867423, Boehringer Mannheim) for tagged proteins and TU-02 (Santa Cruz) for a-tubulin. For analyses of CDK4 protein, we found that the use of the AB-1/DCS-35 antibody was critical because other commercially available antibodies detected cross-reacting non-CDK4 proteins of similar size to CDK4.

### EXAMPLE 3

This example demonstrates that c-MYC directly regulates CDK4 mRNA expression.

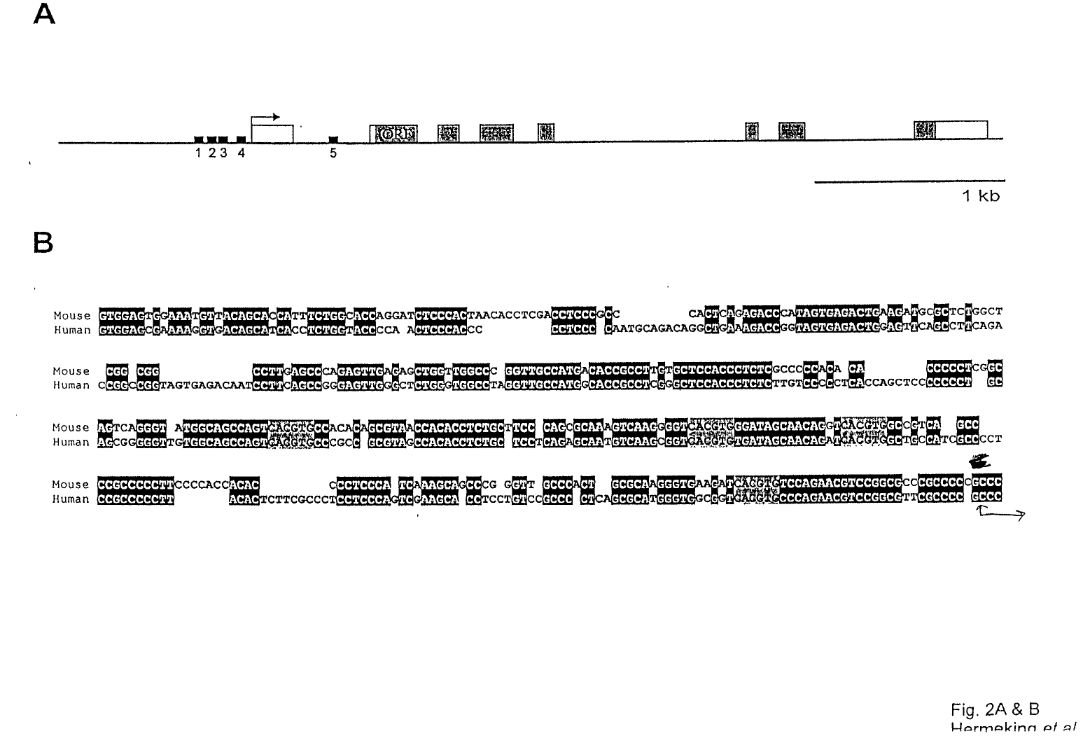

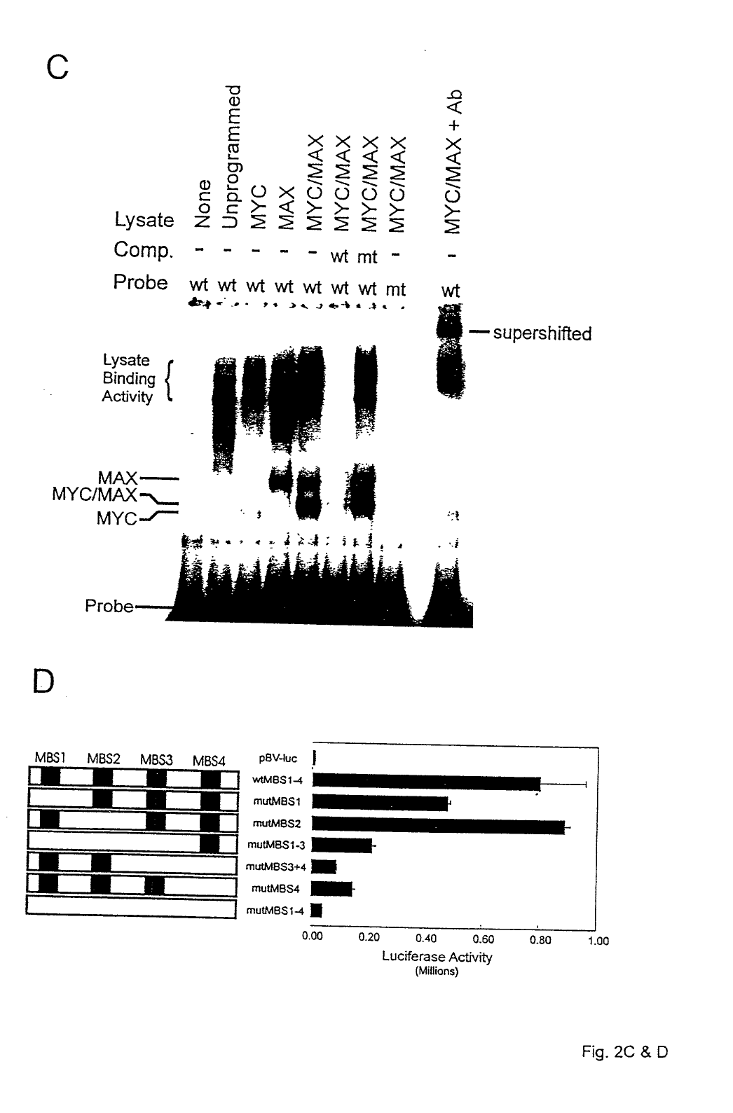

The possibility that c-MYC directly regulates CDK4 mRNA expression was further supported by examination of the human CDK4 gene sequence. There were only five potential c-MYC-binding sites (MBS) within the entire 45,976 bp within and surrounding the CDK4 coding sequence, four of which were clustered in a 200 bp region immediately upstream of the transcription start site (FIG. 2A and B). As the effect of c-MYC expression on human and rodent cell cycle re-entry is similar, MBS would be expected to be present in the murine CDK4 gene promoter if CDK4 were a general target of c-MYC. To evaluate this possibility, we determined the sequence of the murine Cdk4 gene promoter after isolating a mouse BAC containing this gene. Remarkably, the murine promoter contained the same four MBS (MBS1-4), identical to those observed in humans in sequence and in position with respect to the Cdk4 transcription start site (FIG. 2B). MBS5 was not found to be conserved.

To test whether c-MYC actually binds these putative MBS, gel electrophoretic mobility shift assays (EMSA) were performed with the MBS containing portion of the CDK4 promoter. c-MYC/MAX complexes specifically bound a CDK4 promoter fragment containing MBS1-MBS4 but not a CDK4 promoter fragment containing a mutant MBS1-MBS4 in which each MBS had a single nucleotide substitution (CACGTG→CACCTG) (FIG. 2C). The specificity of the observed complexes was demonstrated by competition with wild type CDK4 MBS but not mutant CDK4 MBS (FIG. 2C). Addition of an antibody directed against an HA-epitope present in the recombinant MAX protein was able to generate a “supershift” of the putative MYC/MAX and MAX complexes bound to DNA. Isolation of the Human and Murine CDK4 Genes. The primer pair 5′-CAGCATCACCTCTGGTACCC-3′ and 5′-CCCGAATTCCGGGGCGAACGCCGGACG-3′ respectively, derived from the cosmid sequence ((13) and GenBank HSU81031) containing the CDK4 promoter region was used to screen a human BAC library. A BAC (662M22, Research Genetics) containing the CDK4 promoter was digested with Kpn I. A 2 kb fragment containing the CDK4 promoter was identified using PCR and then subcloned into pBR322 (corrected sequence deposited as GenBANK entry ####). For isolation of the murine cdk4 gene the primer pair 5′-CTGCCACTCGATATGAACCCG-3′ and 5′-TAGATCCTTAATGGTCTCAACCG3′ derived from the mouse Cdk4 cDNA was used to identify a BAC (509, Research Genetics) containing the mouse Cdk4 gene. A 4 kbp Kpn I fragment containing the promoter, exon 1 and 2 and the first intron was then subcloned into pBR322 and partially sequenced (sequence deposited as GenBANK ####).

Gel Electrophoretic Mobility Shift Assays. DNA binding assays were performed in 25 mM Tris HCl (pH 7.5), 80 mM NaCl, 35 mM KCl, 5 mM MgCl₂, 1 mM DTT, 6 ug/ml poly(dIdC), 10% glycerol, 2.4% NP40. Proteins were generated by a coupled in vitro transcription/translation using the TNT T7 Quick System (Promega) and employing Max and ct-Myc (a truncated version of c-MYC) encoding plasmids described in (14). Approximately 10⁶ cpm of end-labeled oligonucleotides (40 ng DNA) was used per reaction. The respective wild type and mutant DNA CDK4 promoter fragments were released by a Kpn I/BamH I digestion from the reporter constructs described below. DNA and proteins were incubated for 30 min at room temperature. Anti-HA antibody (Cat# 1867423, Boehringer Mannheim) was added for the last 15 minutes of this incubation The complete reactions were then loaded on a non-denaturing 5% acrylamide gel and separated in 0.5×TBE (1×=0.1 M Tris pH 8.4, .09 M boric acid, 1 mM EDTA) for 6 hours at 4° C. at 100 V.

### EXAMPLE 4

This example demonstrates that the four potential MBS sequences are required for transactivation of CDK4 by c-MYC.

To test whether the four potential MBS sequences were required for transactivation of CDK4 by c-MYC, reporter-constructs with specific point mutations in the MBS1-4 sequences (CACGTG→CACCTG) were generated in different combinations (FIG. 2D). A fragment encompassing 200 bp of the region directly upstream of the CDK4 transcription start site conferred strong transcriptional activity to a reporter after introduction into RAT1 cells (FIG. 2D). The activity of this reporter was mediated through MBS1-4 sequences, as mutation of all four sites almost completely abrogated transactivation. Mutation of individual MBS elements suggested that MBS3 and MBS4 were particularly important for mediating the c-MYC responsiveness of the CDK4 promoter (FIG. 2D). To further evaluate the c-MYC responsiveness of the CDK4 promotor, we tested the ability of exogenous c-Myc to activate the CDK4 reporters in NIH3T3 fibroblasts (FIG. 2E). These studies indicated that wild-type c-Myc, but not a mutant c-Myc lacking the HLH domain, transactivated the CDK4 promoter by 4 to 5 fold. Point mutations of the four MBS (mutMBS1-4) resulted in a markedly diminished basal activity of the mutant promoter, whose activity remained about 100-fold less active than the wild-type promoter even in the presence of co-transfected wild-type c-Myc. These data suggest that c-Myc directly activates the CDK4 promoter in an E-box dependent manner.

Reporter Assays. To generate reporter constructs, the following oligonucleotides were used:

5′-CCGGTACCGGGTTGTGGCAGCCAGTCACGTGCCCGCCGCGTAGCCACACCTCTGCTCCTCA
GAGCAATGTCAAGCGGTCACGTGTGATAGCAACAGATCACGTGGCTGCCATCGCCCCTC-3′

(Oligo A, for wild type MBS 1-3),
5′-ATGAATTCCGGACGTTCTGGGCACGTGACCGCCACCCATG
CGCTGAGGGGCGGACAGGAGGTGCTTCGACTGGGAGGAGGGCGAAGAGTGTAAGGGGGCGG
AGGGGCGATGGCAGCC-3′
(Oligo B, for wild type MBS 4),
5′-CCGGTACCGGGTTGTGGCAGCCAGTCACCTGCCCGCCGCGTAGCC
ACACCTCTGCTCCTCAGAGCAATGTCAAGCGGTCACCTGTGATAGCAACAGATCACCTGGCTG
CCATCGCCCCTC-3′
(Oligo C, for mutant MBS 1-3), and
5′-ATGAATTCCGGACGTTCTGGGCAGGTGACCGCCACCCATGCGCTGAGGGGCGGACAGGAG
GTGCTTCGACTGGGAGGAGGGCGAAGAGTGTAAGGGGGCGGAGGGGCGATGGCAGCCAGG-3′

(Oligo D, for mutant MBS 4). Different combinations of oligonucleotide pairs (A+B, A+D, C+B, C+D) were annealed and converted to double stranded fragments through 1 PCR cycle. These promotor fragments were subcloned into the Kpn I/BamH I sites of pBV-luc, a modified pGL3 -basic derived reporter containing a minimal promoter (15). Further polymerase-derived mutants (mutMBS2 and mutMBS3+4) were identified while sequencing the reporter constructs. For reporter assays in RAT1 cells, transfections were performed using Lipofectamine (Life Sciences), 1 mg of reporter plasmid and 0.1 mg of a β-galactosidase reporter to control for transfection efficiency. Luciferase and β-galactosidase activities were assessed 24 h following transfection-using reagents from Promega and ICN Pharmaceuticals, respectively. To test the ability of exogenous cMyc to transactivate reporters, subconfluent NIH3T3 fibroblasts were transfected by Lipofectin (Gibco) with 2 mg of reporter plasmid and different amounts of either MLV-LTR driven plasmids expressing wild type c-Myc or mutant c-Myc with the helix-loop-helix (HLH) domain deleted (deletion of amino acids 371-412) (16). Luciferase activity was measured 48 hours after transfection following the manufacturer's protocol (Promega). Total DNA amount was equalized by adding different amounts of empty MLV-LTR vector.

Cell lines: The RAT1 fibroblast subclone TGR-1 and the c-Myc−/−derivatives have been described (6). RAT1 fibroblasts and BOSC23 (7) packaging lines were cultured in growth medium (DMEM supplemented with 10% calf serum, Life Technologies, Gaithersburg, Md.).

Retrovirus Generation. The CDK4 ORF was generated by PCR using the EST W77860 as a template and the primers 5′-GCGGATCCGCGGCCGCCTTCCACCATGGCTACCTCTCGATCTGAGC-3′ and 5′-CGGTCGACTCACTCCGGATTACCTTCATC-3′. The resulting product was digested with the enzymes Not I and Sal I and inserted into the respective sites of the vector G1BgSVNA (a retroviral vector encoding a hygromycin resistance gene and β-galactosidase) replacing the β-galactosidase gene. The unmodified vector was used as a control. Bosc23 packaging cells (7) were transfected and the supernatant of resistant, pooled cells was used to infect Rat1 cells.

### EXAMPLE 5

This example demonstrates the roles and relationships of c-MYC and CDK4 in the Cell Cycle

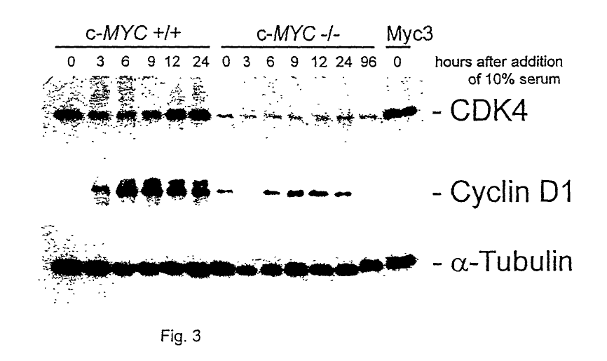

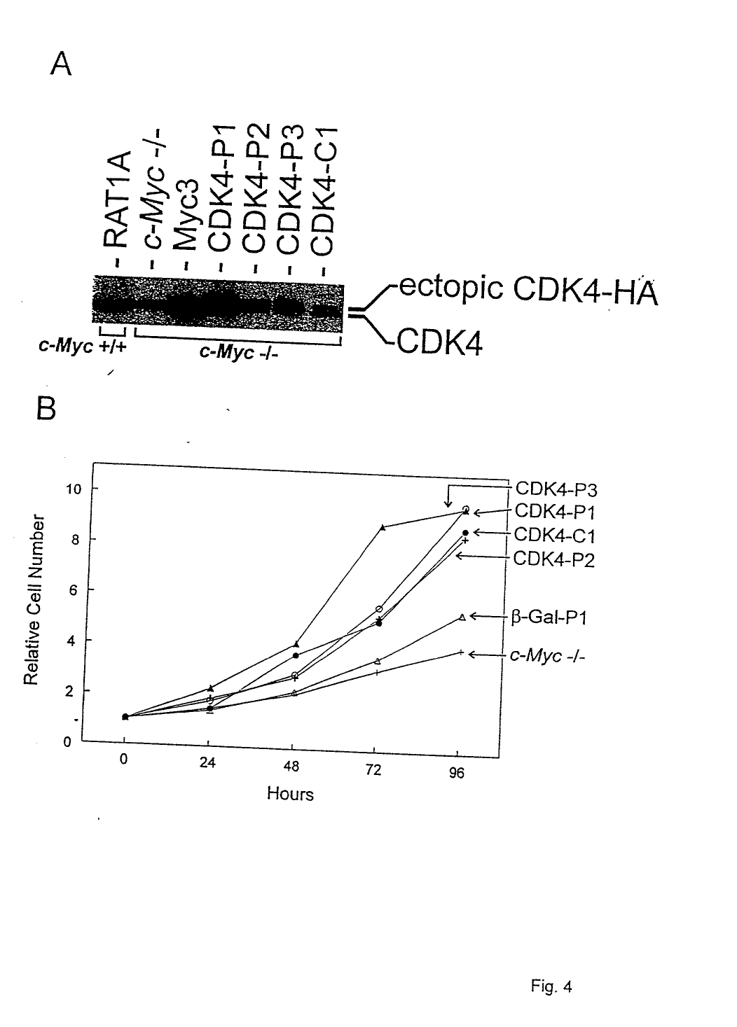

In order to determine whether c-MYC plays a role in the induction of CDK4 by mitogens, we studied Ratl fibroblasts in which the c-Myc gene had been inactivated by homologous recombination (6). These cells exhibit an extension of their G1- and G2-phases, leading to an increase in cell doubling time from 18 hours to approximately 50 hours (25). Serum stimulated induction of Cdk4 mRNA was attenuated and delayed in c-Myc-deficient cells. This attenuation was evident whether normalized for total cellular RNA (FIG. 3A) or cell count (FIG. 3B) and was about two fold greater than the deficit observed for the induction of Gap-DH and other house keeping genes in the c-Myc deficient cells. Consistent with this deficit, both serum starved and exponentially growing c-Myc deficient cells displayed lower basal levels of Cdk4 mRNA than their wild type counterparts (FIG. 3B and data not shown). Additionally, Cdk4 expression was restored in c-Myc-I-cells that ectopically expressed c-Myc from a retroviral construct (FIG. 4A and data not shown). The defect in Cdk4 mRNA induction was also reflected by a defect in induction of Cdk4 protein (FIG. 3C). In contrast to Cdk4, Cyclin D1 showed higher than normal levels of induction after serum stimulation of c-Myc-deficient cells (FIG. 3C) confirming that c-Myc deficient cells do not have a general defect in their mitogenic signaling cascades as previously reported (25).

We next hypothesized that the failure to form active Cdk4/Cyclin D1 complexes contributed to the previously observed prolongation of the G1 -phase in c-Myc deficient Rat1 cells grown in the presence of serum. To test this conjecture, c-Myc−I−Rat-1 cells were infected with retroviruses conferring expression of either CDK4 or β-Galactosidase. Analysis of the CDK4 retrovirus infected cells revealed expression of CDK4 at levels comparable to those seen in wild type Rat1 cells (FIG. 4A). Ectopic CDK4 expression led to a significant increase in growth rate (FIG. 4B). The doubling time of CDK4-expressing c-Myc−I−Rat1 cells was reduced to 29.75 hours (SD 2.3, n=8) when compared to parental or β-Galactosidase expressing cells, which doubled every ˜42.8 (SD 5.27, n=4) hours.

### EXAMPLE 6

This example demonstrates the expression of c-MYC and CDK4 in human Tumors

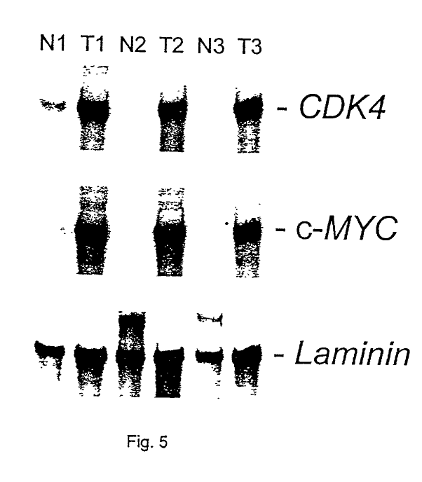

To determine whether the link between c-MYC and CDK4 extends to naturally occurring human tumors, we evaluated colorectal cancers. It has previously been shown that these cancers overexpress c-MYC (e.g., 26, 27), usually because of genetic defects in APC or β-catenin, which regulate the activity of the c-MYC promoter (15). Northern blot analysis revealed a concordant increase in c-MYC and CDK4 expression in colorectal cancers when compared to normal colorectal epithelium derived from the same patients (FIG. 5). This observation was consistent with previous reports showing increases in CDK4 levels in early adenomas of mice and humans with APC mutations (28,29).

Discussion

The above results suggest that the ability of c-MYC to promote cell cycle re-entry is in part due to its ability to directly induce the transcription of CDK4. This mechanism is consistent with several previous observations. First, embryonic fibroblasts derived from Cdk4−I−mice show a prolonged transition from G1 to S-phase after serum stimulation (30,31), similar to the phenotype of c-Myc deficient fibroblasts (6). Second, a striking defect in Cyclin/Cdk activity was recently demonstrated in c-Myc-deficient fibroblasts, with a 12-fold reduction in the activity of Cdk4/Cyclin D1 and Cdk6/Cyclin D1 complexes (25). Our results suggest that one factor contributing to the reduction was the reduced amounts of Cdk4 protein in c-Myc-deficient cells. Because Cdk4 is regulated at multiple levels, it is likely that other Myc-dependent factors also contribute to the defect in Cdk4 activity in c-Myc deficient cells. Indeed, the reduction of Cdk4 activity is significantly greater than the reduction in Cdk4 protein (25 and unpublished data). Third, c-MYC can antagonize the growth inhibition mediated by three different CDK-inhibitors (p21, p27, and p16), suggesting that c-Myc induces a protein that can compensate for such inhibition (21,32,33). CDK4 is a protein that could clearly function in this manner, since it can serve to sequester p21, p27, and p16 (34,35). This sequestration may account for the ability of c-Myc overexpression to substitute for p16 deficiency in mouse fibroblast transformation (36). Finally, a target of CDK4 phosphorylation is the retinoblastoma tumor suppressor gene product pRB (37,38) and as noted above, CDK4 can inhibit the activity of p16. The ability of CDK4 to functionally inactivate the products of two tumor suppressor genes, RB and p16, provides a link between c-MYC and the CDK4/CYCLIN D1/pRB/p16 pathway and may account for the lack of genetic alterations of RB and p16 in some cancers. In such cancers, the elevated c-MYC expression and the consequent elevation of CDK4 expression could obviate the driving force for mutations in RB and p16. Consistent with this model, expression of CDK4 was shown to transform primary REFs (rat embryo fibroblasts) in cooperation with activated Ha-rasG12V(39). Furthermore, ectopic expression of a fusion gene between CDK4 and Cyclin D1 is able to immortalize primary REFs and cooperates with activated Ha-ras to transform REFs conferring anchorage-independent growth in vitro and formation of tumors in vivo (40). Cyclin D1 and Ha-rasG12V coexpression alone did not lead to transformation, suggesting that cdk4 is necessary for transformation and immortalization (40). In these assays, CDK4/Cyclin D1 could be substituted by c-MYC (40). One puzzling observation made in the course of our studies is that Cdk4 transcription was not induced by Myc estrogen receptor (MycER) chimeras in RAT1 cells (data not shown). We do not know whether this is due to a subtle defect in the MycER protein compared to native protein, to physiological alterations in the MycER cell lines, or to a more complex regulation of Cdk4 by c-Myc than suggested by our model.

Transcriptional targets of c-MYC have long been sought. CDK4 is especially interesting for several reasons. The induction of CDK4 was observed following c-MYC expression independent of species (human or mouse) and cell type (endothelial, fibroblast, B-cells or epithelium), albeit to varying degrees. The regulation of CDK4 by c-MYC appeared to be direct, as suggested by the conservation of c-MYC binding sites in the CDK4 promotor and by their ability to confer responsiveness to exogenous MYC in reporter assays. Finally, the experiments reported here, as well as those reviewed above, provide plausible mechanisms that explain how this target (CDK4) can mediate some of the effects of c-MYC on the cell cycle. Though any single target is unlikely to explain all of c-MYC's activities, CDK4 provides a direct link between c-MYC's ability to promote tumorigenesis and cell cycle regulation.

### References

1. Amati, B., Alevizopoulos, K. & Vlach, J. (1998) Front Biosci 3, 250-268.

2. Dang, C. V. (1999)Mol. Cell. Biol. 19, 1-11 .

3. Eick, D. & Hermeking, H. (1996) Trends Genet. 12, 4-6.

4. Garte, S. (1993) Crit. Rev. Oncogenisis 4, 435-449.

5. Eilers, M., Schirm, S. & Bishop, J. M. (1991) Embo J. 10, 133-141.

6. Mateyak, K. M., Obaya, A. J., Adachi, S. & Sedivy, J. M. (1997) Cell Growth Differ. 8, 1039-1048.

7. Pear, W. S., Nolan, G. P., Scott, M. L. & Baltimore, D. (1993) Proc. Natl. Acad Sci. USA 90, 8392-8396.

8. He, T. C., Zhou, S., da Costa, L. T., Yu, J., Kinzler, K. W. & Vogelstein, B. (1998) Proc. Natl. Acad. Sci. USA 95, 2509-2514.

9. Hermeking, H., Wolf, D. A., Kohlhuber, F., Dickmanns, A., Billaud, M., Fanning, E. & Eick, D. (1994) Proc. Natl. Acad. Sci. USA 91, 10412-10416.

10. Berns, K., Hijmans, E. M. & Bernards, R. (1997) Oncogene 15, 1347-1356.

11. Hermeking, H., Lengauer, C., Polyak, K., He, T. C., Zhang, L., Thiagalingam, S., Kinzler, K. W. & Vogelstein, B. (1997) Mol. Cell 1, 3-11.

12. Velculescu, V. E., Zhang, L., Vogelstein, B. & Kinzler, K. W. (1995) Science 270, 484-487.

13. Elkahloun, A. G., Krizman, D. B., Wang, Z., Hofmann, T. A., Roe, B. & Meltzer, P. S. (1997) Genomics 42, 295-301.

14. Kohlhuber, F., Hermeking, H., Graessmann, A. & Eick, D. (1995) J. Biol. Chem. 270, 28797-28805.

15. He, T. C., Sparks, A. B., Rago, C., Hermeking, H., Zawel, L., da Costa, L. T., Morin, P. J., Vogelstein, B. & Kinzler, K. W. (1998) Science 281, 1509-1512.

16. Shim, H., Dolde C., Lewis, B. C., Wu, C. S., Dang, G., Jungmann, R. A., Dalla-Favera, R. & Dang, C. V. (1997) Proc. Natl. Acad Sci. USA 94, 6658-6663.

17. Matsushime, H., Ewen, M. E., Strom, D. K., Kato, J. Y., Hanks, S. K., Roussel, M. F. & Sherr, C. J. (1992) Cell 71, 323-334.

18. Ewen, M. E., Sluss, H. K., Whitehouse, L. L. & Livingston, D. M. (1993) Cell 74, 1009-1020.

19. Alexandrow, M. G., Kawabata, M., Aakre, M. & Moses, H. L. (1995) Proc. Natl. Acad Sci. USA 92, 3239-3243.

20. Latham, K. M., Eastman, S. W., Wong, A & Hinds, P. W. (1996) Mol. Cell. Biol. 16, 4445-4455.

21. Hermeking, H., Funk, J. O., Reichert, M., Ellwart, J. W. & Eick, D. (1995) Oncogene 11, 1409-1415.

22. Wang, J., Xie, L. Y., Allan, S., Beach, D. & Hannon, G. J. (1988) Genes Dev. 12, 1769-1774.

23. Holland, E. C., Hively, W. P., Gallo, V. & Varmus, H. E. (1998) Genes Dev. 12, 3644-3649.

24. Schuhmacher, M., Staege, M. S., Pajic, A., Polack, A., Weidle, U. H., Bornkamm, G. W., Eick, D. & Kohlhuber, F. (1999) Curr. Biol. 9, 1255-1258.

25. Mateyak, M. K., Obaya, A. J. & Sedivy, J. M. (1999) Mol. Cell. Biol. 19, 4672-4683.

26. Erisman, M. D., Rothberg, P. G., Diehl, R. E., Morse, C. C., Spandorfer, J. M. & Astrin, S. M. (1985)Mol. Cell. Biol. 5, 1969-1976.

27. Augenlicht, L. H., Wadler, S., Corner, G., Richards, C., Ryan, L., Multani, A. S., Pathak, S., Benson, A., Haller, D. & Heerdt, B. G. (1997) Cancer Res. 57, 1769-1775.

28. Zhang, T., Nanney, L. B., Luongo, C., Lamps, L., Heppner, K. J., DuBois, R. N. & Beauchamp, R. D. (1997) Cancer Res. 57, 169-175.

29. Zhang, T., Nanney, L. B., Peeler, M. O., Williams, C. S., Lamps, L., Heppner, K. J., DuBois, R. N. & Beauchamp, R. D. (1997) Cancer Res. 57, 1638-1643.

30. Rane, S. G., Dubus, P., Mettus, R. V., Galbreath, E. J., Boden, G., Reddy, E. P., Barbacid, M.S. & Rane, G. (1999) Nat. Genet. 22, 44-52.

31. Tsutsui, T., Hesabi, B., Moons, D. S., Pandolfi, P. P., Hansel, K. S., Koff, A. & Kiyokawa, H. (1999) Mol. Cell. Biol. 19, 7011-7019.

32. Steiner, P., Philipp, A., Lukas, J., Godden-Kent, D., Pagano, M., Mittnacht, S., Bartek, J. & Eilers, M. (1995) Embo J. 14, 4814-4826.

33. Vlach, J., Hennecke, S., Alevizopoulos, K., Conti, D. & Amati, B. (1996) Embo J. 15, 6595-6604.

34. Reynisdottir, I., Polyak, K., lavarone, A. & Massague, J. (1995) Genes Dev. 9, 1831-1845.

35. Serrano, M., Hannon, G. J. & Beach, D. (1993) Nature 366, 704-707.

36. Serrano, M., Lin, A. W., McCurrach, M. E., Beach, D. & Lowe, S. W. (1997) Cell 88, 593-602.

37. Koh, J., Enders, G. H., Dynlacht, B. D. & Harlow, E. (1995) Nature 375, 506-510.

38. Lukas, J., Parry, D., Aagaard, L., Mann, D. J., Bartkova, J., Strauss, M., Peters, G. & Bartek, J. (1995) Nature 375, 503-506.

39. Haas, K., Staller, P., Geisen, C., Bartek, J., Eilers, M. & Moroy, T. (1997) Oncogene 15, 179-192.

40. Rao, R. N., Stamm, N. B., Otto, K., Kovacevic, S., Watkins, S. A., Rutherford, P., Lemke, S., Cocke, K., Beckmann, R. P., Houck, K., Johnson, D. & Skidmore, B. J. (1999) Oncogene 18, 6343-6356.

1. A reporter construct comprising:
an upstream region of a mammalian CDK4 gene transcription start site comprising at least four c-MYC binding sites; and
a coding sequence for a reporter protein, wherein the upstream region is upstream of the coding sequence, and wherein the upstream region and coding sequence are operably linked so that a wild-type c-MYC upon binding to the upstream region activates transcription of the coding sequence.

2. The reporter construct of claim 1 wherein the c-MYC binding site is CACGTG.

3. The reporter construct of claim 1 wherein the region is at least 200 bp.

4. The reporter construct of claim 1 wherein the upstream region comprises a CDK4 promoter.

5. The reporter construct of claim 1 wherein the mammalian CDK4 gene is human CDK4.

6. A host cell comprising:
a reporter construct according to claim 1; and
a c-MYC protein;
wherein the c-MYC protein binds to the reporter construct and activates transcription of the coding sequence for the reporter protein.

7. The host cell of claim 6 which overexpresses c-MYC.

8. A method to screen test compounds for anti-cancer activity, comprising the steps of:
contacting a c-MYC protein with a reporter construct according to claim 1 in the presence of a test compound; and
monitoring expression of the reporter protein;
wherein a test compound which decreases expression of the reporter protein is a candidate anti-cancer agent.

9. The method of claim 8 wherein the reporter construct and the c-MYC protein are in a host cell and the test compound is contacted with the host cell.

10. The method of claim 8 wherein the reporter construct and the C-MYC protein are contacted in a cell-free transcription/translation system.

11. An isolated and purified nucleic acid molecule comprising at least one copy of a region upstream of a human CDK4 gene transcriptional start site, wherein the region comprises at least four c-MYC binding sites comprising the sequence CACGTG, wherein the nucleic acid molecule does not contain the CDK4 coding sequence.

12. The nucleic acid molecule of claim 11 wherein the region comprises at least 200 bp.

13. The nucleic acid molecule of claim 11 which is attached to a solid support.

14. A method to screen test compounds for anti-cancer activity, comprising the steps of:
contacting a c-MYC protein with a nucleic acid molecule according to claim 11 in the presence of a test compound; and
monitoring binding of c-MYC protein to the nucleic acid molecule,
wherein a test compound which decreases binding of c-MYC to the nucleic acid molecule is identified as a candidate anti-cancer agent.

15. A method of inhibiting the growth of tumor cells, comprising the step of:
contacting tumor cells which comprise a genetic alteration which causes c-MYC overexpression with an agent which inhibits CDK4 enzymatic activity, whereby tumor cell growth is inhibited.

16. The method of claim 15 wherein the tumor cells are Burkitt's Lymphoma cells.

17. The method of claim 15 wherein the tumor cells are neuroblastoma cells.

18. The method of claim 15 wherein the tumor cells are colon cancer cells.

19. The method of claim 15 wherein the tumor cells have a t8;14 translocation.

20. The method of claim 15 wherein the tumor cells have a genetic amplification of c-MYC.

21. The method of claim 15 wherein the tumor cells have a mutation in APC.

22. The method of claim 21 wherein the tumor cells have a truncating mutation in APC.

23. The method of claim 15 wherein the agent is p16.

24. The method of claim 15 wherein the agent is a polypeptide comprising a truncated version of p16.

25. A method of screening compounds to identify those which have anti-cancer activity, comprising the step of:
contacting a cell which has a genetic alteration which dysregulates c-MYC expression with a test compound;
measuring activity of CDK4 in the cell, wherein a test compound which inhibits activity of CDK4 is identified as a candidate agent with anti-cancer activity.

26. The method of claim 25 wherein the cell is a Burkitt's Lymphoma cell.

27. The method of claim 25 wherein the cell is a neuroblastoma cell.

28. The method of claim 25 wherein the cell is a colon cancer cell.

29. The method of claim 25 wherein the cell has a t8;14 translocation.

30. The method of claim 25 wherein the cell has a genetic amplification of c-MYC.

31. The method of claim 25 wherein the cell has a mutation in APC.

32. The method of claim 21 wherein the cell has a truncating mutation in APC.

33. A method of determining responsiveness to an anti-cancer agent which inhibits CDK4 activity, comprising:
testing a cancer cell for the presence of a mutation selected from the group consisting of: a t8;14 translocation, an APC mutation, an amplification of c-MYC, and a β-catenin mutation;
wherein a cancer cell which is identified as having said mutation is identified as being susceptible to an inhibitor of CDK4.

34. The method of claim 33 further comprising the step of:
administering to the cancer cell an anti-cancer agent which inhibits CDK4 activity.
